(19) United States
(12) Patent Application Publication
(10) Pub. No.: US 2012/0156151 A1
(43) Pub. Date: Jun. 21, 2012

(54) PIGMENTS AND METHODS OF MAKING PIGMENTS

(76) Inventor: Guilherme L. Indig, Brookfield, WI (US)

(21) Appl. No.: 13/393,089

(22) PCT Filed: Aug. 31, 2010
(86) PCT No.: PCT/US10/47328

§ 371 Date: Feb. 28, 2012

(30) Foreign Application Priority Data
Aug. 31, 2009 (US) 61275535

(51) Int. Cl. A61K 8/19; C08K 3/34; A23L 1/275; C04B 16/00; A61Q 1/02; C04B 33/00; C09D 11/00

(52) U.S. Cl. 424/63; 501/141; 106/487; 106/31.6; 524/445; 106/803; 426/540

(57) ABSTRACT

A method of making a pigment comprising a dye and a clay having pores. The method includes mixing a first reaction component with first and second dye components to form a reaction mixture, wherein the first reaction component comprises the clay and a catalyst associated with the clay, wherein the first and second dye components are adapted to react with each other in the presence of the catalyst to form the dye, and whereupon the catalyst catalyzes the reaction of the first and second dye components, and at least some of the first and second dye components react within the pores.

### CROSS-REFERENCE TO RELATED APPLICATIONS

This patent application claims priority to U.S. Provisional Patent Application No. 61/275,535 filed Aug. 31, 2009, the content of which is incorporated herein by reference in its entirety.

### INTRODUCTION

Pigments are used as colorants for a wide variety of industrial goods including paints, inks, coatings, plastics, textiles, cosmetics, foods, cements, ceramics, and the like. Even in prehistoric times, naturally occurring pigments, such as ochres, iron oxides and indigo pigments, were used as colorants for both aesthetic and artistic purposes. Prior to the industrial revolution, the range of pigment colors available for decorative and artistic uses was limited. Most of these pigments were earth and mineral pigments, or pigments of biological origin. Pigments derived from uncommon sources, such as localized plants, animal wastes, insects and mollusks, were harvested and traded over long distances.

### BRIEF DESCRIPTIONS OF THE DRAWINGS

The present patent or patent application file contains at least one drawing executed in color. Copies of this patent or patent application publication with color drawing(s) will be provided by the Office upon request and payment of the necessary fee.

FIG. 1 is series of graphs comparing powder x-ray data for (A) pure indigo, (B) a physical mixture of pure indigo and pure sepiolite, (C) pure sepiolite, and (D) the indigo-sepiolite pigment composition prepared by the method disclosed herein.

FIG. 2 is a photograph showing pigments made from different dyes and clays.

### DETAILED DESCRIPTION

For example, in early Mesoamerica, the Mayans and Aztecs used an azure blue pigment (later referred to as Maya blue) composed of indigo (an organic dye derived from the leaves of the plant Indigo suffruticosa), and palygorskite (a naturally occurring inorganic clay). When indigo and palygorskite clay are mixed together and heated, they produce a stable pigment with a brilliant blue color similar to that found in Mesoamerica. Remarkably, Maya blue has proven to be resistant to fading over time and under harsh conditions, as evidenced by the fact that Mayan paintings have not faded significantly over time. Maya blue has been tested against biodegradation and chemical aggression (i.e. by treatment with chemical solvents, acids and alkalis), and has been shown to be extremely resistant. However, Maya blue can be destroyed using acid treatment under reflux.

Generally, there is a lack of understanding regarding either the chemistry for producing pigments from dyes and clays, or the structure of such pigments. Clays, such as the palygorskite clay used by the Mayans and Aztecs to produce Maya blue, are composed of inorganic minerals (e.g. aluminum silicates, magnesium and/or iron silicates) that form microscopic crystals having structures that depend on their compositions. The clays include metal ions that may coordinate with other compounds. The clays typically are porous, having microscopic and/or nanoscopic pores arranged in a maze-like network within the crystalline lattice of the clay. Many of these pores are narrower than 1-2 nanometers in diameter. When these clays are mixed with dyes, the dye molecules bind to the outside of the clay particles via hydrogen bonding or electrostatic interactions with the clay and/or by coordination with the metals in the clay. Some dye molecules potentially may diffuse, at least slightly, into relatively larger internal spaces formed by the clay where they may be bound more tightly to the clay than on the outer surface. It is possible that this diffusion may, in part, be facilitated by heating the mixture of the dye and clay at very high temperatures for extended periods of time, as in the conventional processes for making Maya blue. However, due to their size, dye molecules likely have a difficult, if not impossible time diffusing into and through the narrow pores in the clay to reach spaces deep within the crystalline lattice of the clay.

References generally relating to dyes, pigments, color compositions, and/or methods for making the same include: U.S. Pat. Nos. 3,950,180; 5,061,290; 4,246,036; 4,640,862; 4,868,018; 4,874,433; 5,574,081; 5,972,049; 5,993,920; 6,339,084; 6,402,826; 7,052,541; 7,425,235 and 7,429,294, the complete disclosures of which are herein incorporated by reference in their entireties for all purposes.

The present disclosure provides pigments formed of a dye and a clay, and methods for making such pigments. The method may include (a) providing a first reaction component comprising the clay and a catalyst associated with the clay, (b) providing first and second dye components adapted to react with each other in the presence of the catalyst to form the dye, and (c) mixing the first and second dye components with the first reaction component to form a reaction mixture, whereupon the catalyst catalyzes the reaction of the first and second dye components, and at least some of the first and second dye components react within the pores of the clay. As discussed in more detail below, the dye may include any type of dye that is formed by a condensation reaction between one or more dye components in the presence of a catalyst including, but not limited to, dyes formed by Baeyer condensation reactions, dyes formed by Baeyer-Drewson condensation reactions and fluorescent dyes formed by condensation reactions. The clay may include any type of porous clay including, but not limited to, sepiolite, attapulgite, chlorite, illite, montmorillonite, saponite, kaolinte, dickite, halloysite, nacrite, kaglin, bentonite, and palygorskite clays, as well as synthetic clays such as alumilite and laponite, among others. The catalyst may include an acid or base, a reducing or oxidizing agent (e.g. a metal), an organic material, or any other suitable catalyst, and either may be intrinsic to the chemical composition of the clay, or may be mixed with the clay prior to mixing the clay with the dye components.

The methods disclosed herein may facilitate the formation of highly stable pigments due to entrapment of dye within the pores of the clay. Specifically, upon mixing the small dye precursors (i.e., the first and second dye components) with the clay, the dye precursors may diffuse into and through the small pores in the crystalline lattice of the clay. When these dye precursors react within the pores to form the larger dye molecules, they may be entrapped within the clay, similar to a ship that has been assembled in a bottle. Pigments having dye entrapped within the clay may have different properties (e.g., better stability, different color, etc.) than pigments formed by other methods. For example, pigments having dye entrapped within the clay may be more stable to at least one of UV light, heat, caustics, etc.

Some of the dye produced upon condensation of the dye precursors also may bind to the outside of the clay particles. For example, some of the dye precursors may react outside of the pores to form dye that is not entrapped within the clay, and some of this non-entrapped dye may bind to the outer surfaces of the clay via hydrogen bonding or electrostatic interactions with the clay and/or by coordination with the metals in the clay. Dye coordination sites may have their origin either in metal ions naturally present in the clay, or in selected metal ions added to the clay via treatment with solutions of metal ions, and the metal composition of the clay may affect the color of the pigment.

The methods of the present disclosure may be at least one of faster, more environmentally friendly and less expensive than conventional processes for making pigments. Conventional methods of making pigments from dyes and clays generally include (a) making a dye; (b) mixing the dye with clay, and (c) heating the mixture at high temperatures for long periods of time, such as for several days. Making a dye alone often requires multiple processing steps and the use of excessive quantities of organic solvents and/or caustic chemicals, particularly on a commercial scale. The present method does not require a separate step for making a dye prior to mixing with the clay, and thus does not require the costly processing steps or chemicals necessary for making such a dye. Moreover, the present method generally either can be performed at room temperature, or can include microwaving, sonicating, and/or slightly heating the reaction mixture for a short period of time to increase the rate of pigment formation.

A wide variety of differently colored pigments may be formed by the method disclosed herein, and these pigments may be useful as colorants for many different industrial goods including paints, inks, coatings, plastics, textiles, cosmetics, foods, cements, and the like. As discussed below, the properties, including the stability and color, of a selected pigment may depend on the selected dye, the selected clay, the conditions for catalyzing the reaction between the dye precursors in the presence of the clay, and various post-processing steps.

I. Clays

The clay may include any type of porous clay. Examples may include, but are not limited to at least one of sepiolite, attapulgite, chlorite, illite, montmorillonite (e.g., montmorillonite K10 or montmorillonite KSF, etc.), saponite, kaolinte, dickite, halloysite, nacrite, kaglin, bentonite, and palygorskite clays, as well synthetic clays such as alumilite and laponite, and combinations thereof, among others. Porous clays generally comprise aluminum, magnesium and/or iron silicates that form microscopic crystals having structures that depend on their compositions. The metals in the clays may coordinate with other compounds, and other functional groups in the clay may form hydrogen or electrostatic bonds with other compounds. The pores may be microscopic and/or nanoscopic, and may be arranged in a maze-like network within the crystalline lattice of the clay. Some pores may be narrower than 1-2 nanometers in diameter. The clays generally may be acidic, having Brønsted acids dispersed throughout.

Using different clays in the presently disclosed method may create pigments having different properties, such as different colors or stabilities to external factors. These properties may depend on the particle size of the clay, the pore size of the clay, the metal composition of the clay, the acidity/alkalinity of the clay, etc.

The particle size of the clay may be varied, such as using any grinding or milling technique known in the art. The particle sizes may be at least about 0.01 μm, at least about 0.05 μm or at least about 0.1 μm. The particle sizes may be less than about 20 μm, less than about 10 μm or less than about 1 μm. For example, the particle size may be between about 0.01 μm and about 20 μm, between about 0.05 μm and about 10 μm, or between about 0.1 μm and about 1 μm.

II. Catalysts

The catalyst may be intrinsic to the chemical composition of the clay, or may be mixed with the clay prior to mixing the clay with the dye components. For example, for pigments made of dyes formed by acid-catalyzed condensation reactions, the catalyst may be the acid that is intrinsic to the clay itself, which may include Brønsted and/or Lewis acids. For pigments made of dyes formed by base-catalyzed condensation reactions, the clay may be mixed with a base (such as NaOH) to form a basic clay prior to mixing the clay with the dye precursors. Likewise, for pigments made of dyes formed by condensation reactions that are catalyzed by some other catalyst (e.g. a metal or metal-based catalyst), the catalyst either may be intrinsic to the clay or the clay may be mixed or treated with that catalyst prior to mixing the clay with the corresponding dye precursors.

III. Dyes

The pigments disclosed herein may include any dye that is formed by a condensation reaction between one or more dye components in the presence of a catalyst, including but not limited to a Brønsted acid, a Lewis acid, a weak or strong base, a metal or metal-based catalyst, etc. Suitable dyes may include, but are not limited to, dyes formed by Baeyer-Drewson condensation reactions, dyes formed by Baeyer condensation reactions, fluorescent dyes formed by condensation reactions, and/or any suitable combination of dyes.

Dyes formed by Baeyer-Drewson condensation reactions may include, but are not limited to, indigo and derivatives of indigo such as dibromoindigo, thioindigo, sulfonated indigo or any other indigo derivative known or hereafter discovered or devised. As shown in Scheme I, the classical Baeyer-Drewson reaction involves forming indigo by treating a mixture of o-nitrobenzaldehyde and acetone with aqueous NaOH (i.e. a base-catalyzed reaction).

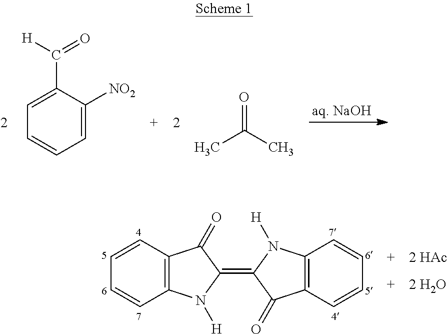

Dyes formed by a Baeyer condensation reaction may include, but are not limited to, any triarylmethanes, such as crystal violet, malachite green, brilliant green, ethyl violet, methyl green, basic red 9, basic violet 14, methyl violet 2b, Hoffman's violet, Victoria blue R, Victoria blue B, Victoria pure blue BO, and/or any other suitable triarylmethane. Scheme II shows various reactions that may be carried out in organic solvents in the presence of an acid catalyst to form the reduced (i.e. leuco, or colorless) form of triarylmethane dyes, which subsequently need to be oxidized to form the colored product. In conventional processes, this oxidization step generally includes the use of organic oxidizing agents, such as chloranil. It should be appreciated that triarylmethanes may be formed by reacting substantially any aromatic aldehyde or substituted aromatic aldehyde with one or more aromatic amines or substituted aromatic amines, although any known precursor compounds may be used.

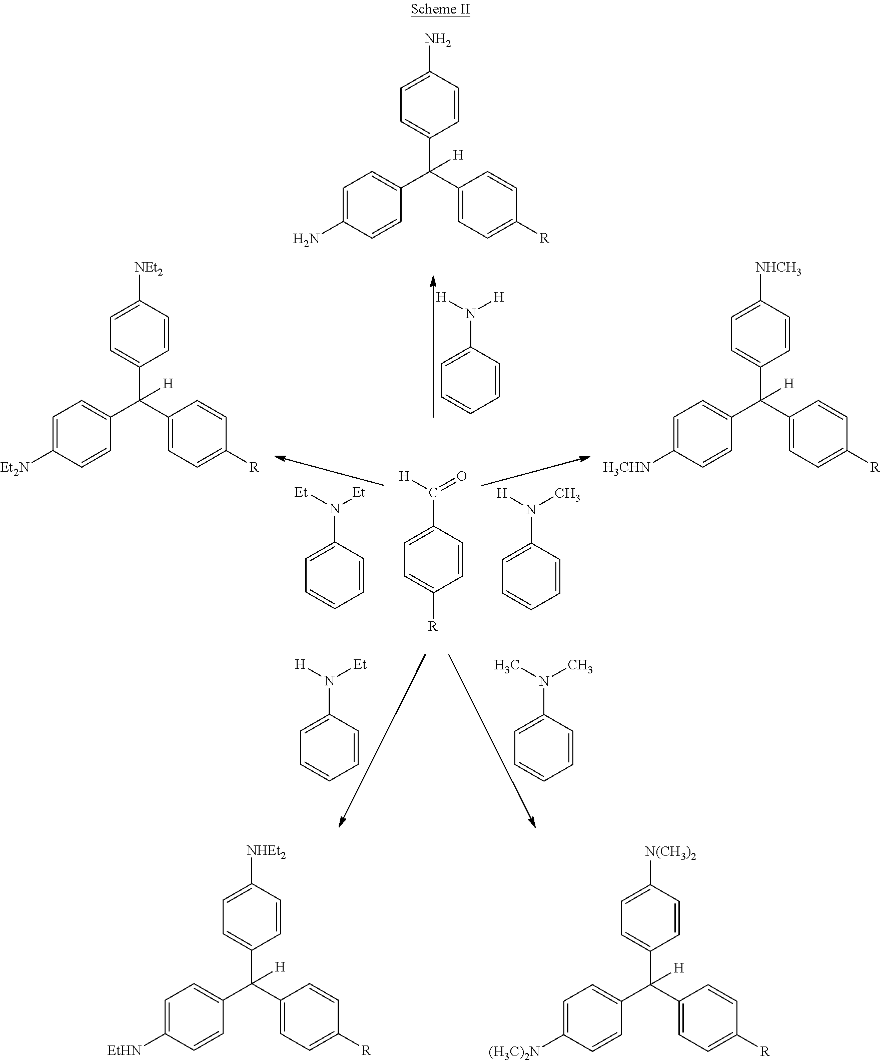

Fluorescent dyes formed by a condensation reaction may include, but are not limited to, xanthene dyes, such as fluorescein and fluorescein derivatives, rhodamine and rhodamine derivatives, etc. Scheme III shows the acid-catalyzed condensation reaction for synthesizing xanthene dyes, where X, for example, may be —OH (fluorescein), NH₂ (rhodamine 110), or N(CH₂CH₃)₂ (rhodamine B).

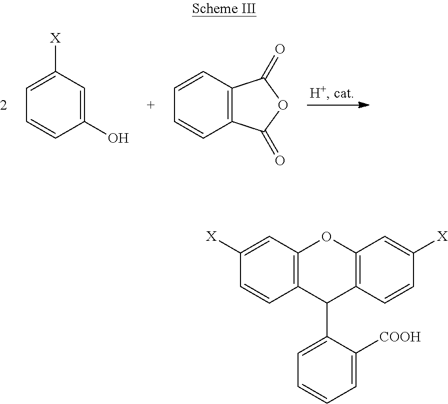

Any other dye formed by a condensation reaction may be used to make a pigment according to the method disclosed herein. In some cases, a mixture of multiple dyes also may be used.

IV. Methods of Making the Pigments

As discussed above, the present method of making pigment generally includes (a) providing a first reaction component comprising a clay and a catalyst associated with the clay, (b) providing first and second dye components adapted to react with each other in the presence of the catalyst to form a dye, and (c) mixing the first and second dye components with the first reaction component to form a reaction mixture, whereupon the catalyst catalyzes the reaction of the first and second dye components, and at least some of the first and second dye components react within the pores of the clay. This general method may be used to make a wide variety of pigments with different properties. The properties (e.g., the stability, color, etc.) of a selected pigment depends on the selected dye, the selected clay, the conditions for catalyzing the reaction between the dye precursors in the presence of the clay, and various post-processing steps. As such, the specifics associated with making any particular pigment depends, in large part, on the selected dye and the chemistry for forming that dye.

The various dyes that can be formed by a Baeyer-Drewson condensation reaction can be used to form a wide variety of differently colored pigments using the method disclosed herein. In order to make pigments from dyes formed by a Baeyer-Drewson condensation reaction, a first reaction component may be prepared by mixing a selected clay (which is generally acidic) with a strong base, such as NaOH. A second reaction component may be prepared by mixing the dye precursors of the Baeyer-Drewson reaction (e.g. o-nitrobenzaldehyde or a derivative thereof and acetone) with one another in the absence of any base. Because the Baeyer-Drewson condensation reaction is base-catalyzed, mixing the first reaction component (i.e. the base treated clay) with the second reaction component (i.e. the mixture of the dye precursors in the absence of base) effectively catalyzes the reaction between the dye precursors only once they are in the presence of the clay. At least some of the dye precursors may react within pores in the clay, thereby entrapping the dye in the pores. The colors of the pigments made with dyes formed by Baeyer-Drewson condensation reactions may depend on the selected dye, the relative amounts of the dye precursors in the reaction mixture, the selected clay, the relative amount of clay in the reaction mixture, the alkalinity of the clay, and/or any post-condensation reaction processing steps.

The various dyes that can be formed by Baeyer condensation reactions also can be used to form a wide variety of differently colored pigments using the method disclosed herein. For example, a first reaction component may be provided that includes a clay having a Brønsted acid intrinsic to the chemical composition of the clay. Because Baeyer condensation reactions generally are acid-catalyzed reactions, the first reaction component may be mixed with the dye precursors (e.g. first and second dye components, such as those shown in Scheme II) thereby catalyzing the reaction between the dye precursors only once they are in the presence of the clay. At least some of the dye precursors may react within pores in the clay, thereby entrapping the dye in the pores. The colors of the pigments made with dyes formed by Baeyer condensation reactions may depend on the selected dye, the relative amounts of the dye precursors in the reaction mixture, the selected clay, the relative amount of clay in the reaction mixture, the acidity of the clay, and/or any post-condensation reaction processing steps.

Fluorescent dyes that can be formed by condensation reactions also can be used to form a wide variety of different pigments using the method disclosed herein. For example, a first reaction component may be provided that includes a clay having a Brønsted acid intrinsic to the chemical composition of the clay. The dye precursors of the acid-catalyzed condensation reaction (e.g. first and second dye components, such as those shown in Scheme III) may be mixed with the first reaction component, thereby catalyzing the reaction between the dye precursors only once they are in the presence of the clay. At least some of the dye precursors may react within pores in the clay, thereby entrapping the dye in the pores. The colors of these pigments may depend on the selected dye, the relative amount of dye precursors in the reaction mixture, the selected clay, the relative amount of clay in the reaction mixture, etc.

For many dyes, the general method disclosed herein can be used to quickly form pigments at room temperature. For example, in some cases, pigment formation may be completed in less than about 120 minutes, less than about 60 minutes, less than about 30 minutes, less than about 15 minutes, less than about 10 minutes, less than about 5 minutes, or even less than about 1 minute. Depending on the selected dye, some additional steps, such as microwaving, heating and/or sonicating the reaction mixture, may be used to increase the rate of pigment formation and/or facilitate the formation of the pigment (as evidenced by the rate of color formation). For example, some dye precursors may be difficult to solubilize, in which case the dye precursors may be difficult to mix in a manner that facilitates reaction. In such cases, the dye precursors may be heated or microwaved for a short period of time (e.g. between about 2 and about 20 minutes) to melt the dye precursors and facilitate their reaction to form the dye and pigment. Examples of dyes that may require such treatment include some fluorescent dyes that may be difficult to solubilize. Microwaving, heating and/or sonicating the reaction mixture for short periods of time also may increase the vibrational, translational and/or rotational energy states of the dye precursors, which may facilitate diffusion of the dye precursors into the pores of the clay and reaction of the dye precursors with each other. It has been observed that even short periods of microwaving, heating and/or sonicating the reaction mixture may be sufficient to facilitate pigment formation when used in combination with the general method disclosed herein. For example, some reactions may be facilitated by sonicating, microwaving and/or heating the reaction mixture at temperatures less than about 100° C. for at least about 1 minute, at least about 2 minutes, at least about 5 minutes, or at least about 10 minutes. Some reaction mixtures may be facilitated by sonicating, microwaving and/or heating the reaction mixture less than about 120 minutes, less than about 60 minutes, less than about 30 minutes, or less than about 15 minutes. Some reactions may be facilitated by sonicating, microwaving and/or heating the reaction between about 1 minute and about 120 minutes, such as between about 2 minutes and about 60 minutes, between about 5 minutes and about 30 minutes, or between about 10 minutes and about 15 minutes. Regardless of whether pigment formation occurs at room temperature, or is facilitated by short periods of heating, microwaving or sonicating, the present method can be performed substantially faster than conventional methods, and requires the use of substantially less heat (e.g. lower temperatures for shorter periods of time).

After the condensation reaction has proceeded to completion, additional steps may be used to process the pigment, include filtering, washing and/or drying the pigment. For example, after the condensation reaction has proceeded to completion, the reaction mixture may include excess unreacted reagents and/or impurities that would be desirable to remove from the final pigment. In such cases, the pigments may be washed (e.g. with water, detergents, acids/bases, organic solvents, oxidizing/reducing agents, etc.), filtered, and/or dried to provide the final pigment. In some cases, these steps may change the color and/or structure of the pigment. For example, the oxidation state of some dyes (and thus the color of the dye and corresponding pigment) may be changed by washing the pigment with an oxidizing or reducing agent including, but not limited to, ascorbic acid, formic acid, hydrogen peroxide, metal salts, nitric acid, nitrous oxide, oxalic acid, silver oxide, or any other suitable oxidizing or reducing agent. The dye and the corresponding pigment also may be chemically altered by treatment with certain chemicals, such as strong acids and bases or any other suitable chemical.

V. Color Compositions Formed with the Pigments

The pigments disclosed herein can be used to form a wide variety of different color compositions, including but not limited to, paints, inks, coatings, plastics, textiles, cosmetics, foods, cements, ceramics, etc. To form such color compounds, the pigment may be mixed with other compounds, including polymers, binding agents, solvents, emulsifiers, modifiers, or any other suitable compound. These compounds may increase at least one of the stability, uniformity, spreadability, adherability, thickness, etc. of the color composition and/or the pigment in the color composition. These compounds are well known, and vary broadly, depending on the particular application for the color compound.

For example, if the pigment is to be used as a colorant for a paint, it may be mixed with binding agents that may include acryl, acrylate, acryl acid butyl esters, acrylonitrile polymers, butyl rubbers, chlorosulphonated polyethylene, chlorosulphonated ethylene-acrylic acid copolymers, cyclized rubber, ethylstyrene-acrylonitrile copolymers, hydrocarbon resins, methacrylate, methylstyrene-acrylonite copolymers, polyacrylic acid esters, polyamides, polyesters, polyester imide polyethylene, polyethylene, polyethylene oxidate, poliisopropyl acrylate, polyurethanes, aliphatic polyurethanes and polyolefins, polyvinylpyrrolidone, styrene acrylate, vinylidene chloride copolymer, vinylpyrrolidone-vinyl acetate copolymer, and/or any other suitable binding agent. The pigment also may be blended with a variety of other compounds, including but not limited to gum Arabic, linseed oil, copal, polycarbonate, egg tempura, and turpentine, among many others, to create blended systems. The color of the blended color composition may then be altered depending on the medium in which it is blended. Moreover, the pigment may be ground to the desired particle size prior to and/or during blending to affect the color.

It is to be understood that the methods and compositions contemplated and supported by the present disclosure are not limited to the specific methods and compositions set forth herein. The invention is capable of other embodiments and of being practiced or of being carried out in various ways. Also it is to be understood that the phraseology and terminology used herein is for the purpose of description only, and should not be regarded as limiting. Ordinal indicators, such as first, second, and third, as used in the description and the claims to refer to various structures, are not meant to be construed to indicate any specific structures, or any particular order or configuration to such structures. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification, and no structures shown in the drawings, should be construed as indicating that any non-claimed element is essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

Further, no admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinency of any of the documents cited herein.

### EXAMPLES

The following examples are included to demonstrate preferred embodiments of the methods and compositions disclosed herein. These examples are merely illustrative and in no way limit the scope of any claimed invention.

### Example 1

### Method of Making Pigments Comprising Dyes Formed by Baeyer-Drewson Condensation Reactions

A first reaction component was prepared by homogenizing 250 g of sepiolite clay with 2.5 L of 0.5 M aqueous NaOH. A second reaction component was prepared by mixing 450 ml of acetone (i.e. a first dye component) and 63.3 g of 2-nitrobenzaldehyde (i.e. a second dye component). The second reaction component (i.e. the mixture of the first and second dye components in the absence of catalyst) was slowly added to the first reaction component (i.e. the base treated clay) under conditions of vigorous blending to form a reaction mixture, whereupon the NaOH catalyst catalyzed the reaction between the dye acetone and the 2-nitrobenzaldehyde. This base-catalyzed reaction between the first and second dye components was exothermic, and was complete within 2 minutes, after which mixing was stopped. The reaction mixture containing the sepiolite clay, the reacted indigo, and any unreacted dye components (e.g. acetone) was left to cool for approximately 30 minutes, during which time some of the excess acetone evaporated. After the Baeyer-Drewson condensation reaction was complete and the reaction mixture was allowed to cool, the pigment formed by the reaction was separated from other remaining components of the reaction mixture. It was determined that this separation could be performed in several different ways. In some cases, the reaction mixture was mixed with 2.5 L of water and dialyzed against distilled water to neutral pH. The slurry was then dried at 120° C., leaving a dry blue pigment. In some cases, the reaction mixture was neutralized with HCl, filtered, washed multiple times with water, and then dried, leaving a dry blue pigment. In some cases, the reaction mixture was washed with water, washed with acetone, and then dried, leaving a dry blue pigment.

Other pigments were similarly prepared with dyes formed by Baeyer-Drewson reactions and either bentonite or montmorillonite KSF clays instead of sepiolite clay. 1.0 g of bentonite and 1.0 g of montmorillonite KSF were placed in distinct glass vials and briefly exposed (2-3 minutes) to 10 ml of a 1.0 M sodium hydroxide solution under continuous stirring. The excess liquid phase was removed from each sample by filtration, and the respective moist clays were placed back into distinct reaction vials and exposed to ultrasound and vigorous mechanical stirring while 1.0 ml of a 0.53 M solution of 2-nitrobenzaldehyde in acetone was added drop wise to each vial. Color formation (i.e. formation of the indigo dye via the base-catalyzed condensation reaction of the dye components) was observed within approximately 30 seconds of contact of the organic reaction component (i.e. the mixture of 2-nitrobenzaldehyde and acetone in the absence of catalyst) and inorganic reaction component (i.e. the clay treated with NaOH) at room temperature. The reaction mixtures were homogenized, filtered, washed with lukewarm water, and dried, leaving distinct blue pigments. It should be appreciated that other means for separating the pigment from remaining components of the reaction mixture may be used.

Another organic-inorganic pigment composite was prepared with Tyrian purple (i.e., 6,6′-dibromoindigo) and sepiolite clay. Tyrian purple dye may be formed by the Baeyer-Drewson condensation reaction of 4-bromo-2-nitrobenzaldehyde with acetone in the presence of an alkaline catalyst. To form the pigment, a first reaction component was prepared by mixing 5.0 g of sepiolite clay with 35 ml of 0.1 M aqueous NaOH. A second reaction component was prepared by mixing 6 ml of acetone with 80 mg of 4-bromo-2-nitrobenzaldehyde in the absence of base. The second reaction component (i.e. the mixture of the first and second dye components) was added to the first reaction component (i.e. the alkaline treated clay) under vigorous mixing to form a reaction mixture. The reaction mixture was incubated for 30 minutes at room temperature without mixing. The pigment composite formed during the reaction was separated from the other components of the reaction mixture by washing with water, washing with acetone, and drying at room temperature. The pigment formed with Tyrian purple dye and sepiolite clay was purple, and had a significantly different color than pigments prepared with indigo dye and sepiolite clay.

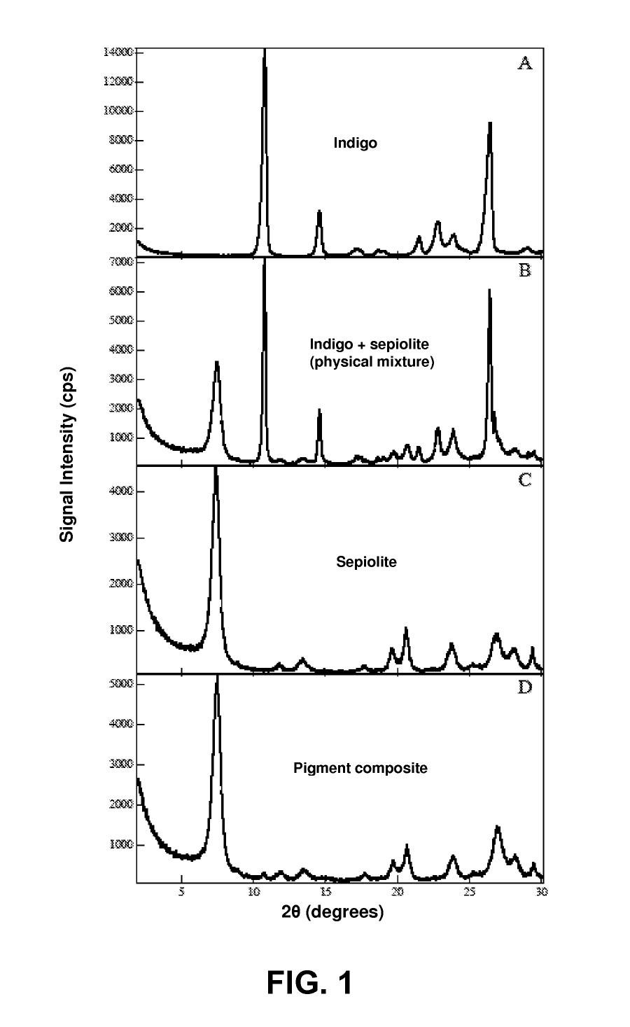

X-ray powder diffraction was utilized to compare the crystal structure of the indigo-sepiolite pigment composite described above with the crystal structures of a simple physical mixture of indigo and sepiolite, indigo alone, and sepiolite alone. For these studies, all samples were homogenized to comparable powder appearances with a mortar and pestle. The powder X-ray data is shown in FIG. 1, where FIG. 1A shows the X-ray spectrum of indigo alone, FIG. 1B shows the X-ray spectrum of a physical mixture of indigo and sepiolite, FIG. 1C shows the spectrum of just sepiolite, and FIG. 1D shows the spectrum of the indigo-sepiolite pigment composite. The peaks shown in FIG. 1A (indigo alone) and FIG. 1B (physical mixture of indigo+sepiolite) at 2θ≈11, 14 and 27 degrees indicate the presence of solid (crystalline) indigo. Notably, these peaks are not observed in FIGS. 1C (sepiolite alone) or FIG. 1D (indigo-sepiolite pigment composite). The X-ray spectrum for the indigo-sepiolite pigment composite is most similar to that of the pure sepiolite clay. This supports that the indigo in the indigo-sepiolite composite pigment is largely dispersed throughout all regions of the clay to which the respective small organic dye precursors had access, such as within the nanoscopic pores of the clay.

Stability assays (or resistance to destruction assays) were performed on the indigo-sepiolite composite pigment in the presence of concentrated nitric acid. The stability of the composite pigment was directly compared with physical mixtures of indigo and sepiolite clay, as well as heat-treated samples of those mixtures. First, two physical mixtures of sepiolite and indigo containing either 20% or 5% by weight of indigo were prepared via mixing of the respective powders with the help of a mortar and pestle. Visually, the 20% mixture was a significantly darker blue color than the indigo-sepiolite composite pigment prepared by the method discussed above, whereas the 5% physical mixture was a lighter blue color than the indigo-sepiolite composite pigment. A portion of the 20% physical mixture was heated at 120° C. for 26 hours to promote adhesion of the indigo to the clay, similar to methods described by others for preparing Maya blue. A portion of the 5% physical mixture was heated at 120° C. for 2 hours, also to promote adhesion. After heating, five separate samples had been prepared: (a) the 20% physical mixture, (b) the 20% physical mixture heated for 26 hours at 120° C., (c) the 5% physical mixture, (d) the 5% physical mixture heated at 120° C. for 2 hours, and (e) the indigo-sepiolite pigment prepared by the method discussed above. 50 mg of each of these samples was exposed to 4 ml of concentrated nitric acid and subjected to vigorous magnetic stirring for 15 minutes. Each sample was subsequently filtered and washed with distilled water.

While the final blue color of the indigo-sepiolite composite pigment appeared as a lighter blue color after nitric acid treatment as compared to the color of the original material, this sample was the least affected by acid treatment upon visual inspection. The colors of the 20% and 5% physical mixtures that were not subjected to heat treatment changed from blue to tan with a very faint green hue after nitric acid treatment. The colors of the heated 20% physical mixture changed from a dark blue to a light green-grey, while the color of the heated 5% physical mixture changed from a lighter blue to a tan-green color by visual inspection. As such, the indigo-sepiolite composite pigment retained significantly more color than the other samples after treatment with concentrated nitric acid. These results support that the indigo-sepiolite composite pigment prepared by the method discussed above is significantly more resistant to extreme acidic conditions (i.e. concentrated nitric acid) than the physical mixtures of sepiolite and indigo, as well as the heat treated mixtures of sepiolite and indigo, thus supporting that the indigo in the indigo-sepiolite composite pigment is at least partially entrapped within the clay (e.g. within the nanoscopic pores in the clay).

An assay for solvent fastness (solvent stability) also was performed on the indigo-sepiolite pigment composite by an independent company (Clariant). Samples of indigo-sepiolite pigment powder were transferred to folded fine pore filters, closed, and immersed in solvents (i.e. ethyl acetate, xylene, acetone, ethanol and 1-methoxy-2-propanol). These test tubes were sealed with cork stoppers and stored at room temperature (23° C.) for 24 hours. The coloration of the solvents was compared with the scale of standard solutions (iodine/potassium iodide solutions). The color fastness of the pigment was evaluated on a scale from 1 (low) to 5 (high). The indigo-sepiolite pigment composite scored 4 out of 5 in this fastness assay as compared to a standard indigo pigment, which scored 2.5 out of 5. These results support that the indigo-sepiolite pigment composite prepared by the method discussed above is more stable than indigo pigment.

### Example 2

### Method of Making Pigments Comprising Dyes Formed by Baeyer Condensation Reactions

As discussed above, numerous Baeyer condensation reactions involving aromatic aldehydes and aromatic amines or other aromatic compounds can be used in the preparation of organic-inorganic pigment composites. In one example, a green pigment may be formed from montmorillonite KSF clay and malachite green dye. Leuco-malachite green dye (colorless) can be formed by a Baeyer condensation reaction between benzaldehyde and N,N-dimethylaniline in the presence of a Brønsted acid catalyst. Leuco-malachite green is then subsequently oxidized throughout the clay template to produce the colored compound malachite green.

To create a malachite green-montmorillonite KSF pigment composite, a first reaction component is provided that includes 1.0 g of montmorillonite KSF clay having a Brønsted acid catalyst associated with the clay (Brønsted acids are intrinsic to the chemical composition of montmorillonite KSF clay). The first reaction component was exposed to 634 μL of N,N-dimethylaniline (the first dye component) and 204 μL of benzaldehyde (the second dye component) at room temperature (−21° C.) in a small glass vial to form a reaction mixture containing both the first and second dye components, the clay and the catalyst. Shortly after mixing (within 10 minutes), this reaction mixture displayed a light green color, indicating a reaction had occurred between dye components. This color of the reaction mixture developed into a darker hue of green after sitting at room temperature for approximately 14-16 hours after mixing. Comparable color intensity was achieved in approximately 4 hours when the reaction mixture was minimally heated at 40° C. Comparable color intensity also was achieved when a 900 W microwave oven was used to expose the reaction mixture to microwaves for even brief periods of time, such as approximately 4 minutes. After the color of the reaction mixture stabilized, which indicated that the reaction had achieved equilibrium, the malachite green-montmorillonite KSF pigment composite that was formed during the reaction was separated from any other reaction mixture components by washing with lukewarm water, filtering, and subsequently drying the pigment. The malachite green-montmorillonite KSF pigment composite formed by this method was green.

Other colored pigments were prepared by treating the pigment with other chemical agents. For example, the malachite green-montmorillonite KSF pigment composite described above was subsequently exposed for ˜1-2 minutes to an aqueous 0.25 M solution of the oxidizing agent FeCl₃, and was subsequently washed to remove excess iron chloride. The FeCl₃-treated malachite green-montmorillonite KSF pigment composite showed a significantly darker green hue as compared to the pigment produced without Fe³⁺ treatment. This supports that, under the experimental conditions described above, not all leuco-malachite green (colorless) originally generated throughout the clay template was subsequently oxidized to the final colored dye solely through the chemistry of the natural montmorillonite KSF clay or exposure to air. It also suggests that this excess (“non-washable”) leuco-malachite green that remains to react with FeCl₃ is entrapped within the pores of the clay and/or is coordinated throughout the clay template. This color darkening effect also is observed upon the addition of FeCl₃ solution to malachite green-montmorillonite KSF pigment composite even after the pigment is washed with ethyl acetate (a solvent in which leuco-malachite green is highly soluble). Again, this supports that the leuco-malachite green formed by the condensation reaction is entrapped within the pores and/or tightly coordinated to the clay.

The final color intensity of malachite green-montmorillonite pigment was found to be controllable (up to color saturation) by changing the relative stoichiometry of the clay and dye components (i.e. clay “loading”). For example, exposure of 1.0 g of montmorillonite KSF to either ½ or ¼ of the amounts of benzaldehyde and/or N,N-dimethylaniline described above led to the formation of pigments showing lighter green colors as compared to the original pigment.

The final color of malachite green-montmorillonite pigment also was modified by post-condensation reaction processing of the originally washed material with acids or bases. Brief treatment (˜1-2 minutes) of the originally prepared malachite green-montmorillonite KSF pigment composite with a 0.5 M aqueous solution of NaOH changes the color of the organic-inorganic pigment composite from its original green to blue. Analogous treatment of the originally prepared malachite green-montmorillonite KSF pigment composite with a 1.0 M solution of HCl leads to a final pigment showing darker green color as compared to the original material.

Numerous other pigments of different colors were prepared with dyes formed by Baeyer condensation reactions. For example, a purple pigment was formed by mixing 1.0 g of montmorillonite KSF clay with 507 μL of aniline and 204 μl of benzaldehyde, and microwaving this reaction mixture for 12 minutes in a conventional 900 W microwave oven. A red pigment was formed by briefly washing this purple pigment with a 0.5 M NaOH. A similar purple pigment was prepared by heating the reaction mixture containing 1.0 g of montmorillonite KSF clay, 507 μL of aniline and 204 μl of benzaldehyde overnight at 75° C. A dark green pigment was formed by preparing a reaction mixture containing 10 g of montmorillonite KSF clay, 6.3 mL of N,N-dimethylaniline, and 2.8 g of p-dimethylamino benzaldehyde, and incubating the reaction mixture overnight at room temperature. A similar dark green pigment was formed by preparing a reaction mixture containing 1 g of montmorillonite KSF clay, 0.63 mL of N,N-dimethylaniline, and 0.28 g of p-dimethylamino benzaldehyde, and briefly microwaving the reaction mixture for about 3 minutes.

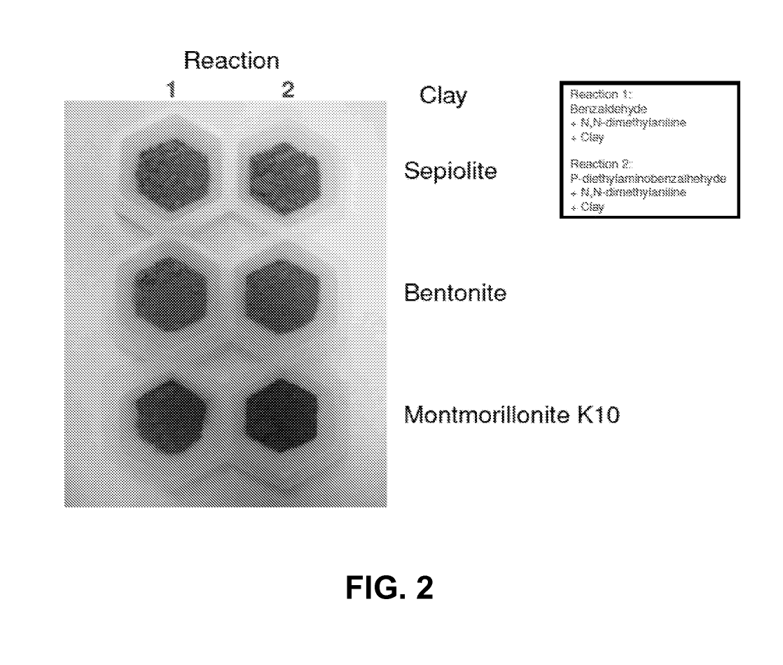

A variety of distinct colors can be formed using the described methods for making organic-inorganic pigment composites by changing the clay used as the inorganic template. As shown in FIG. 2, various blue, gray, green, and brown pigments were made by mixing sepiolite, bentonite, and montmorillonite K10 clays with either N,N-dimethylaniline and benzaldehyde or p-dimethylamino benzaldehyde and N,N-dimethylaniline according to the methods described above. These reaction mixtures were exposed to brief periods of microwave irradiation (about 10 minutes in a conventional 900 W microwave oven) to facilitate (i.e. increase the rate of) color formation. The pigments formed by these reactions were subsequently washed with lukewarm water, filtered, and dried. As shown in column 1 (i.e., “Reaction 1”) of FIG. 2, mixing N,N-dimethylaniline with benzaldehyde in the presence of sepiolite, bentonite and montmorillonite K10 clays produced light blue, avocado green and dark blue pigments, respectively. As shown in column 2 (i.e., “Reaction 2) of FIG. 2., mixing N,N-dimethylaniline with a p-dimethylamino benzaldehyde in the presence of sepiolite, bentonite and montmorillonite K10 clays produced gray-purple, yellow-green and red-brown pigments, respectively. These results illustrate the diversity of final pigment color that can be formed simply by using different clays.

The dark green composite pigment produced via microwave irradiation of the mixture containing montmorillonite KSF, N,N-dimethylaniline and p-dimethylamino benzaldehyde described above (hereafter described as the crystal-violet-montmorillonite KSF pigment composite) was analyzed to assess its stability to strongly alkaline conditions. First, 1×10⁻³ M or 1×10⁻⁴ M aqueous solutions of crystal violet dye were prepared. 15 ml of each of these fully prepared dye solutions was mixed with 2.0 g of montmorillonite KSF to form separate control samples, and these mixtures were washed with water and dried. The stability of the crystal-violet-montmorillonite KSF pigment composite in 3.0 M NaOH was compared to the stability of each control sample in 3.0 M NaOH. Upon exposure to NaOH, the control samples immediately turned violet, whereas the crystal-violet-montmorillonite KSF pigment composite remained green. The samples were allowed to incubate in the NaOH overnight with stirring, and the next day, the control samples remained violet while the sample containing the crystal-violet-montmorillonite KSF pigment remained green. This difference in color stability observed during NaOH treatment supports that the crystal-violet-montmorillonite KSF pigment composite has different properties than pigment formed by mixing a fully formed dye with a clay, and that the crystal violet dye forms and is entrapped within the pores of the clay template.

### Example 3

### Method of Making Stable Pigments Comprising Fluorescent Dyes Formed by Condensation Reaction

Fluorescein may be prepared by the Brønsted acid catalyzed condensation reaction of phthalic anhydride and resorcinol, and was used to make stable pigments with dye reacting within the pores of a clay. A first reaction component was provided that includes approximately 1.0 g of montmorillonite KSF clay having a Brønsted acid catalyst associated with the clay (Brønsted acids are intrinsic to the chemical composition of montmorillonite KSF clay). The first reaction component was mixed with 0.55 grams of solid resorcinol and 0.37 grams of solid phthalic anhydride, and this solid mixture was homogenized with a mortar and pestle. The homogenized solid mixture was transferred to a small capped glass vial and microwaved in a 900 W microwave oven for 2.5 minutes. This caused the resorcinol and phthalic anhydride to melt and thereby react in the presence of the acid catalyst. The final reaction mixture was washed with 0.5 M sodium hydroxide, washed with water, and allowed to dry to generate a yellow fluorescein-montmorillonite composite pigment.

### Example 4

### Pigment Color Can be Controlled by the Type of Dye Used

As discussed above and shown in Table 1 below, a wide variety of stable pigments having different colors can be formed using various condensation reactions, such as Baeyer condensation, Baeyer-Drewson condensation, and condensation reactions forming fluorescent dyes.

TABLE 1

| First Dye | Second Dye | | | Pigment |
|---|---|---|---|---|
| Component | Component | Final Dye | Clay | Color |
| N,N-dimeth- | Benzaldehyde | Malachite | Montmorillonite | Olive |
| ylaniline | | Green | KSF | Green |
| N,N-dimeth- | p- | Crystal | Montmorillonite | Green |
| ylaniline | dimethylamino | Violet | KSF | |
| | benzaldehyde | | | |
| Aniline | Benzaldehyde | No | Montmorillonite | Purple |
| | | Common | KSF | |
| | | Name | | |
| Benzene | Benzaldehyde | Triphenyl | Montmorillonite | Pinkish |
| | | Methane | KSF | Tan |
| Toluene | Benzaldehyde | No | Montmorillonite | Tan |
| | | Common | KSF | |
| | | Name | | |
| Acetone | 2-nitro | Indigo | Montmorillonite | Blue |
| | benzaldehyde | | KSF | |
| Phthalic | Resorcinol | Fluorescein | Montmorillonite | Yellow |
| anhydride | | | KSF | |

### Example 5

### Pigment Color Can be Controlled by the Type of Clay Used as a Starting Template

As discussed above and shown in Table 2 below, pigment color can be controlled by using different clays.

TABLE 2

| First Dye | Second Dye | Final | | Pigment |
|---|---|---|---|---|
| Component | Component | Dye | Clay | Color |
| N,N-dimeth- | Benzaldehyde | Malachite | Montmorillonite | Olive |
| ylaniline | | Green | KSF | Green |
| N,N-dimeth- | Benzaldehyde | Malachite | Sepiolite | Light |
| ylaniline | | Green | | Blue |
| N,N-dimeth- | Benzaldehyde | Malachite | Bentonite | Avocado |
| ylaniline | | Green | | Green |
| N,N-dimeth- | Benzaldehyde | Malachite | Montmorillonite | Dark |
| ylaniline | | Green | K10 | Blue |
| N,N-dimeth- | p- | Crystal | Montmorillonite | Green |
| ylaniline | dimethylamino | Violet | KSF | |
| | benzaldehyde | | | |
| N,N-dimeth- | p- | Crystal | Sepiolite | Gray- |
| ylaniline | dimethylamino | Violet | | Purple |
| | benzaldehyde | | | |
| N,N-dimeth- | p- | Crystal | Bentonite | Yellow |
| ylaniline | dimethylamino | Violet | | Green |
| | benzaldehyde | | | |
| N,N-dimeth- | p- | Crystal | Montmorillonite | Red |
| ylaniline | dimethylamino | Violet | K10 | Brown |
| | benzaldehyde | | | |

### Example 6

### Pigment Color Can be Controlled Using Post Condensation Reaction Processing. For Experimental Details, See Example 1

As discussed above and shown in Table 3 below, pigment color can be controlled using post-condensation reaction processing of the pigments.

TABLE 3

| | | | | Post- | |
|---|---|---|---|---|---|
| | | | | condensation | |
| First Dye | Second Dye | Final | | reaction | Pigment |
| Component | Component | Dye | Clay | processing | Color |
| N,N- | Benzaldehyde | Malachite | Montmorillonite | None | Olive |
| dimethylaniline | | Green | KSF | | Green |
| N,N- | Benzaldehyde | Malachite | Montmorillonite | FeCl₃ | Darker |
| dimethylaniline | | Green | KSF | oxidizing | Olive |
| | | | | | Green |
| N,N- | Benzaldehyde | Malachite | Montmorillonite | NaOH | Blue |
| dimethylaniline | | Green | KSF | | |
| Aniline | Benzaldehyde | No | Montmorillonite | None | Purple |
| | | Common | KSF | | |
| | | Name | | | |
| Aniline | Benzaldehyde | No | Montmorillonite | NaOH | Red |
| | | Common | KSF | | |
| | | Name | | | |

1. A method of making a pigment comprising a dye and a clay having pores, the method comprising:
mixing a first reaction component with first and second dye components to form a reaction mixture, wherein the first reaction component comprises the clay and a catalyst associated with the clay, wherein the first and second dye components are adapted to react with each other in the presence of the catalyst to form the dye, and whereupon the catalyst catalyzes the reaction of the first and second dye components, and at least some of the first and second dye components react within the pores.

2. The method of claim 1, wherein the step of mixing the first reaction component with the first and second dye components comprises:
mixing the first and second dye components to form a second reaction component; and
mixing the first and second reaction components.

3. The method of claim 1, wherein the catalyst comprises a Brønsted acid intrinsic to the chemical composition of the clay.

4. The method of claim 1, wherein the catalyst is a base, and the step of providing the first reaction component includes mixing the clay with the base.

5. The method of claim 1, wherein at least some of the first and second dye components react outside of the pores to form dye that coordinates with coordination sites on an outside surface of the clay.

6. The method of claim 1, further comprising subjecting the reaction mixture to microwave radiation.

7. The method of claim 1, further comprising sonicating the reaction mixture.

8. The method of claim 1, further comprising at least one of the steps of filtering, washing and drying the pigment.

9. The method of claim 8, further comprising treating the pigment with an oxidizing agent, a reducing agent, an acid, a base or an organic solvent.

10. A pigment comprising a dye and a clay having pores, the pigment prepared according to a method comprising:
mixing a first reaction component with first and second dye components to form a reaction mixture, wherein the first reaction component comprises the clay and a catalyst associated with the clay, wherein the first and second dye components are adapted to react with each other in the presence of the catalyst to form the dye, and whereupon the catalyst catalyzes the reaction of the first and second dye components, and at least some of the first and second dye components react within the pores.

11. The pigment of claim 10, wherein the step of mixing the first reaction component with the first and second dye components comprises:
mixing the first and second dye components to form a second reaction component; and
mixing the first and second reaction components.

12. The pigment of claim 10, wherein the catalyst is a Brønsted acid intrinsic to the chemical composition of the clay.

13. The pigment of claim 10, wherein the catalyst is a base, and the step of providing the first reaction component includes mixing the clay with the base.

14. The pigment of claim 10, wherein at least some of the first and second dye components react outside of the pores to form dye that coordinates with coordination sites on an outside surface of the clay.

15. The pigment of claim 10, further comprising subjecting the reaction mixture to microwave radiation.

16. The pigment of claim 10, further comprising sonicating the reaction mixture.

17. The pigment of claim 10, further comprising at least one of the steps of filtering, washing and drying the pigment.

18. The pigment of claim 17, further comprising treating the pigment with an oxidizing agent, a reducing agent, an acid, a base or an organic solvent.

19. A colored composition comprising the pigment of claim 10 and a binding agent.

20. The colored composition of claim 19, wherein the colored composition is a paint, ink, coating, plastic, textile, cosmetic, food, cement or ceramic.

21. A pigment comprising:
a dye; and
a clay having pores;
wherein at least some of the dye is formed by a condensation reaction occurring within the pores.

23. A colored composition comprising the pigment of claim 21 and a binding agent.

24. The colored composition of claim 23, wherein the colored composition is a paint, ink, coating, plastic, textile, cosmetic, food, cement or ceramic.
